(19) United States
(12) Patent Application Publication
(10) Pub. No.: US 2009/0138023 A1
(43) Pub. Date: May 28, 2009

(54) Actuator Handle for Use With Medical Device Deployment Systems

(76) Inventors: Kirk L. Johnson, Weston, FL (US); Juan A. Lorenzo, Davie, FL (US); Robert Lulo, Pembroke Pines, FL (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/086,156

(22) PCT Filed: Dec. 12, 2006
(86) PCT No.: PCT/US2006/061923

§ 371 Date: Jun. 6, 2008

Related U.S. Application Data
(60) Provisional application No. 60/749,830, filed on Dec. 13, 2005.

(51) Int. Cl. A61B 17/00

(52) U.S. Cl. 606/108

(57) ABSTRACT

An actuator handle for use with an implantable medical device deployment system. The actuator handle includes a first actuator and a second actuator for manipulating and controlling first and second retaining members of the deployment system to effectuate release of a medical device from the deployment system.

This application claims the benefit of U.S. Provisional Patent Application No. 60/749,830, filed Dec. 13, 2005, which is hereby incorporated herein by reference.

### FIELD OF THE INVENTION

This invention generally relates to actuator handles for use with medical device deployment systems. More specifically, this invention generally relates to actuator handles for manipulating the movement of retaining members of the deployment system to effectuate the release of a medical device from a deployment system at a target location within the human body.

### DESCRIPTION OF RELATED ART

The use of catheter delivery systems for positioning and deploying therapeutic devices, such as dilation balloons, stents and embolic coils, in the vasculature of the human body has become a standard procedure for treating endovascular diseases. It has been found that such devices are particularly useful in treating areas where traditional operational procedures are impossible or pose a great risk to the patient, for example in the treatment of aneurysms in cranial blood vessels. Due to the delicate tissue surrounding cranial blood vessels, especially for example brain tissue, it is very difficult and often risky to perform surgical procedures to treat defects of the cranial blood vessels. Advancements in catheter deployment systems have provided an alternative treatment in such cases. Some of the advantages of catheter delivery systems are that they provide methods for treating blood vessels by an approach that has been found to reduce the risk of trauma to the surrounding tissue, and they also allow for treatment of blood vessels that in the past would have been considered inoperable.

Typically, these procedures involve inserting the distal end of a delivery catheter into the vasculature of a patient and guiding it through the vasculature to a predetermined delivery site. An implantable medical device, such as an embolic coil or vascular stent, is attached to the end of a delivery member which pushes the medical device through the catheter and out of the distal end of the catheter into the delivery site. Some of the delivery systems associated with these procedures utilize an elongated retaining member, such as a control wire or pull wire, to activate the release and deployment of the medical device. For example, U.S. Pat. No. 5,250,071 to Palermo, which is hereby incorporated herein by reference, describes a deployment system whereby interlocking clasps of the system and the coil are held together by a retaining member. The retaining member is moved proximally to disengage the clasps from each other and release the embolic coil.

Additionally, U.S. patent application Ser. No. 11/461,245, filed Jul. 31, 2006, to Mitelburg, et al., which is hereby incorporated herein by reference for its disclosure of a distal-portion detachment mechanism with which the present invention can be utilized, describes a deployment system wherein a first retaining member engages a hook or eyelet of a second retaining member to attach a medical device to the deployment system. The first retaining member is moved in a proximal direction to disengage it from the hook and release the medical device.

There remains a need for mechanisms or methods for controlling and manipulating retaining members of various medical device deployment systems so as to provide quick and timely deployment of the implantable medical device at a target location within a body vessel. Included is a need for approaches that achieve movement of multiple components to perform desired retaining member functions.

### SUMMARY OF THE INVENTION

In accordance with one embodiment or aspect of the present inventions a handle for use with an implantable medical device deployment system includes a first retaining member and a second retaining member wherein manipulation of the first retaining member and the second retaining member controls the release of an implantable medical device from the deployment system. The handle comprises a handle body adapted to be connected to the deployment system and a first actuator slidably connected to the handle body and extending in a first direction from the handle body. The first actuator can be operatively connected to the first retaining member so that the first retaining member can be manipulated by movement of the first actuator. The handle also includes a second actuator slidably connected to the handle body and extending in a second direction from the handle body. The second actuator can be operatively connected to the second retaining member so that the second retaining member can be manipulated by movement of the second actuator.

In accordance with a further embodiment or aspect of the present invention, a handle for use with an implantable medical device deployment system includes a first retaining member and a second retaining member wherein manipulation of the first retaining member and the second retaining member controls the release of an implantable medical device from the deployment system. The handle comprises a handle body adapted to be connected to the deployment system, and a first actuator slidably connected to the handle body. The first actuator can be operatively connected to the first retaining member so that the first retaining member can be manipulated by movement of the first actuator. The handle also includes a second actuator slidably connected to the handle body. The second actuator can be operatively connected to the second retaining member so that the second retaining member can be manipulated by movement of the second actuator. Additionally, the handle includes a locking mechanism preventing the first actuator from moving relative to the handle body until the locking mechanism is deactivated.

In accordance with yet another embodiment or aspect, a deployment system for delivering an implantable medical device to a target location of a body vessel is provided. The deployment system comprises a generally elongated hollow carrier member having a proximal end portion and a distal end portion, and a first retaining member extending through the hollow carrier member wherein the first retaining member has a proximal end portion and a distal end portion. The deployment system also includes a second retaining member extending through the hollow carrier member wherein the second retaining member has a proximal end portion and a distal end portion. The distal ends of the first and second retaining members cooperating to releasably attach a medical device to the deployment system at near the distal end portion of the carrier member, and manipulation of either of the first retaining member, the second retaining member or both retaining members causes the release of the medical device. The deployment system also includes a handle that has a proximal end portion and a distal end portion, wherein the distal end portion of the handle is connected to the proximal end portion of the carrier member. The handle also includes a first actuator slidably connected to the handle and extending from the handle wherein the first actuator is operatively connected to the first retaining member so that the first retaining member is manipulated by movement of the first actuator. The handle further includes a second actuator slidably connected to the handle and extending from the handle wherein the second actuator is operatively connected to the second retaining member so that the second retaining member is manipulated by movement of the second actuator.

In accordance with a yet another embodiment or aspect, a deployment system for delivering an implantable medical device to a target location of a body vessel is provided. The deployment system comprises a generally elongated hollow carrier member having a proximal end portion and a distal end portion, and a first retaining member extending through the hollow carrier member. The first retaining member has a proximal end portion and a distal end portion. The deployment system also includes a second retaining member extending through the hollow carrier member wherein the second retaining member has a proximal end portion and a distal end portion. The distal ends of the first and second retaining members cooperate to releasably attach a medical device to the deployment system at or near the distal end portion of the carrier member wherein manipulation of either of the first retaining member, the second retaining member or both retaining members causes the release of the medical device. The deployment system also includes a handle that has a proximal end portion and a distal end portion wherein the distal end portion of the handle is connected to the proximal end portion of the carrier member The handle includes a first actuator slidably connected to the handle wherein the first actuator is operatively connected to the first retaining member so that the first retaining member is manipulated by movement of the first actuator. The handle also includes a second actuator slidably connected to the handle wherein the second actuator is operatively connected to the second retaining member so that the second retaining member is manipulated by movement of the second actuator. Additionally, the handle includes a locking mechanism preventing the first actuator from moving until the locking mechanism is deactivated.

In accordance with a yet another embodiment or aspect is directed to a method of deploying an implantable medical device to a target location of a body vessel. The method comprises providing a deployment system having a generally elongated hollow carrier member including a proximal end portion and a distal end portion. The deployment system also includes a first retaining member and a second retaining member each extending through the hollow carrier member. The first and second retaining members cooperating to releasably attach a medical device to the deployment system wherein manipulation of the first and second retaining members effectuate release of the medical device. The deployment system further including a handle connected to the proximal end portion of the carrier member and a first actuator slidably connected to the handle. The first actuator is also connected to the first retaining member for manipulation of the first retaining member. The handle also includes a second actuator slidably connected to the handle wherein the second actuator is connected to the second retaining member for manipulation of the second retaining member. The method further comprising positioning the implantable medical device generally adjacent to a target location within the body vessel. Moving the first actuator to manipulate the first retaining member, and moving the second actuator to manipulate the second retaining member, thereby releasing the medical device.

### BRIEF DESCRIPTION OF THE DRAWINGS

FIG. 1 is a cross-sectional view of an implantable medical device deployment system utilizing one embodiment of an actuator handle in accordance with the present invention in this illustrated release mechanism embodiment;

FIG. 1A is a front perspective view of the distal end of the carrier member of FIG. 1 with portions broken away to show the engagement between the first and second retaining members;

FIG. 2 is a cross-sectional view of the deployment system of FIG. 1, shown with the actuator handle in a partially actuated position;

FIG. 3 is a cross-sectional view of the deployment system of FIG. 1, shown with the actuator handle in the fully actuated position to release the medical device from the deployment system;

FIG. 4 is a cross-sectional view of an implantable medical device deployment system utilizing another embodiment of an actuator handle in accordance with the present invention;

FIG. 5 is a cross-sectional view of the deployment system of FIG. 4 shown with the actuator handle in an unlocked partially actuated position;

FIG. 6 is a cross-sectional view of the deployment system of FIG. 4 shown with the actuator handle in the fully actuated position to release the medical device from the deployment system;

FIG. 7 is a partial cross-sectional view of an implantable medical device deployment system utilizing yet another embodiment of an actuator handle in accordance with the present invention;

FIG. 8 is a partial cross-sectional view of the deployment system of FIG. 7 shown with the actuator handle in an unlocked partially actuated position; and

FIG. 9 is a partial cross-sectional view of the deployment system of FIG. 7 shown with the actuator handle in the fully actuated position to release the medical device.

### DESCRIPTION OF THE PREFERRED EMBODIMENTS

As required, detailed embodiments of the present invention are disclosed herein; however, it is to be understood that the disclosed embodiments are merely exemplary of the invention, which may be embodied in various forms. Therefore, specific details disclosed herein are not to be interpreted as limiting, but merely as a basis for the claims and as a representative basis for teaching one skilled in the art to variously employ the present invention in virtually any appropriate manner.

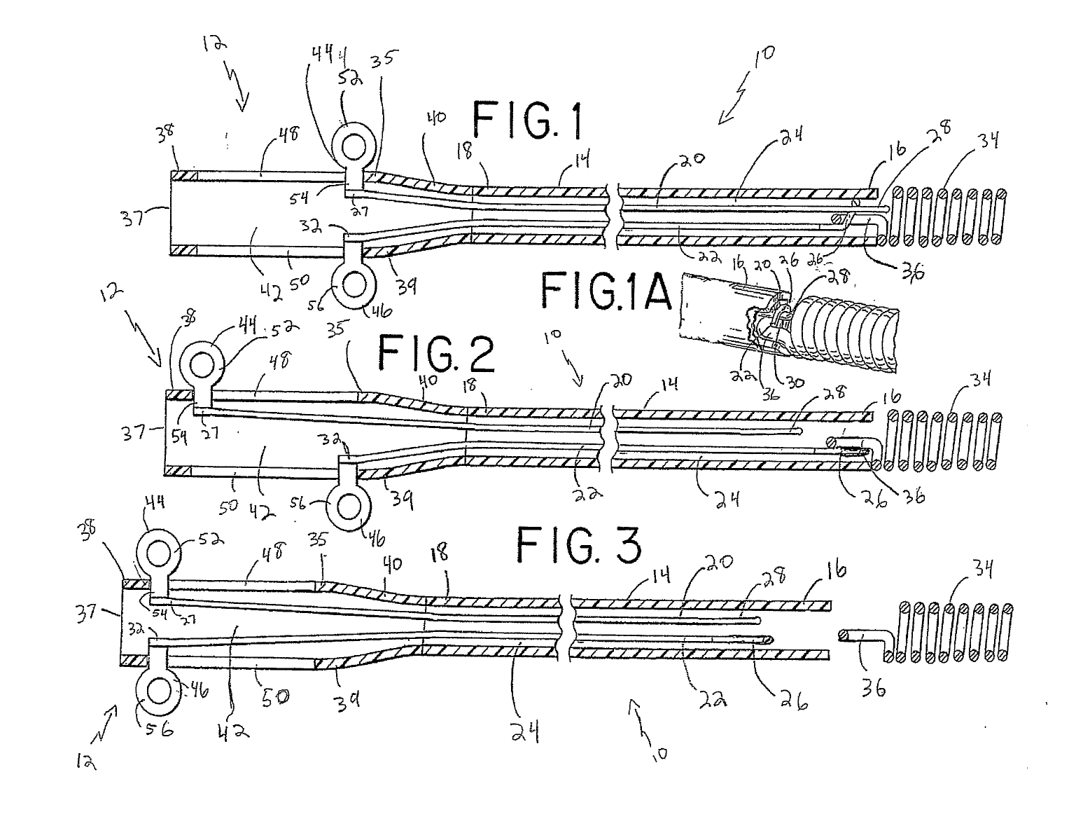

FIGS. 1, 2 and 3 illustrate an implantable medical device deployment system, generally designated at 10, utilizing one embodiment of an actuator handle, generally designated at 12, in accordance with the present invention. The deployment system 10 is generally similar to the deployment system disclosed in U.S. patent application Ser. No. 11/461,245, filed Jul. 31, 2006, to Mitelburg et al., which has been incorporated by reference. However, it will be understood that the actuator handle of the present invention can be used in conjunction with various types of deployment systems, having various configurations, features and release and engagement mechanism. The actuator handles of the illustrated embodiments are especially well suited for deployment systems that have multiple components to be actuated.

The deployment system 10 is comprised of a generally hollow elongated carrier member or pusher 14 having a distal end portion 16 and a proximal end portion 18. Preferably, the carrier member 14 is a hypotube that may be comprised of a biocompatible material, such as stainless steel. The hypotube typically will have a diameter of between about 0.010 inch (0.254 mm) and about 0.015 inch (0.381 mm), a preferred tube having a diameter of approximately 0.013 inch (0.330 mm). Such a carrier member 14 is suitable for delivering and deploying implantable medical devices, such as embolic coils, vascular stents or the like, to target locations, typically aneurysms, within the neurovasculature, but differently sized carrier members comprised of other materials may be useful for different applications.

A first retaining member 20 and a second retaining member 22 extend within lumen 24 of the carrier member 14. The second retaining member 22 has a distal end portion 26 that is associated with the distal end portion 16 of the carrier member 14. The second retaining member 22 can comprise an elongated wire having its distal end length loosely bent in half to define an opening 30 (FIG. 1A) at the distal end portion 26 of the second retaining member 22, The proximal end or ends 32 of the second retaining member 22 extend beyond the proximal end portion 18 of the carrier member 14 so that same can be connected to handle 12.

In one alternative embodiment, the second retaining member 22 can comprise a flat ribbon defining the opening 30 at the distal portion 26. In either embodiment, the second retaining member 22 is preferably deformable to the up-turned condition illustrated in FIGS. 1 and 1A. Additionally, the second retaining member 22 can be elastically deformable to the up-turned condition of FIGS. 1 and 1A, such that it will return to a substantially flat condition, illustrated in FIGS. 2 and 3, when not otherwise constrained, as will be explained in more detail below.

The first and second retaining members 20, 22 may be wires comprised of any of a number of materials, including nitinol, and preferably, are sufficiently stiff to be advanced and/or retracted within the lumen 24 of the carrier member 14. The function of the first and second retaining members 20, 22 will be described in greater detail herein.

As shown in FIGS. 1 and 1A, an implantable medical device 34, such as the illustrated embolic coil, is releasably attached to deployment system 10 by the engagement or cooperation between the distal end portion 28 of the first retaining member 20 and the distal end portion 26 of the second retaining member 22.

To connect the implantable medical device 34 to the deployment system 10, an aperture-containing proximal end portion 36 of the implantable medical device 34 is placed adjacent to opening 30 of the second retaining member 22, which is then deformed to the up-turned condition of FIGS. 1 and 1A. Alternatively, the opening 30 may be moved to the up-turned condition prior to placement of the implantable medical device 34. In the up-turned condition, at least a portion of the opening 30 passes through the aperture of the proximal end portion 36.

As described herein, the second retaining member 22 can be elastically deformable to the up-turned condition of FIGS. 1 and 1A so it will tend to return to a substantially flat condition as illustrated in FIGS. 2 and 3. In order to prevent this, and to consequently attach the implantable medical device 34 to the deployment system 10, the distal end portion 28 of the first retaining member 20 is moved axially through the opening 30 to the position shown in FIGS. 1 and 1A. In this connected condition, the first retaining member 20 holds the second retaining member 22 in the up-turned condition, and the engagement or cooperation between the first and second retaining members 20, 22 releasably secures the proximal end portion 36 of the implantable medical device 34 to the distal end portion 16 of the carrier member 14.

The actuator handle 12 can be comprised of a handle body 35 including a proximal wall 37 and a circumferential wall 39. The circumferential wall can comprise a continuous wall to form a cylinder-like handle body or can be comprised of a series of sub-walls to form a body with a rectangular cross-section. The handle 12 includes a proximal end portion 38 and a distal end portion 40. The distal end portion 40 of the handle 12 is connected to the proximal end portion 18 of the carrier member 14. The first and second retaining members 20, 22 extend past the proximal end portion 18 of the carrier member and into a cavity 42 of the handle 12.

The handle 12 includes a first actuator 44 and a second actuator 46 for manipulating and controlling the movement of the first and second retaining members 20, 22, respectively. The first actuator 44 is slidably connected to a first guide channel 48 in circumferential wall 39, and the second actuator 46 is slidably connected to a second guide channel 50 in wall 39. The first and second guide channels 48, 50 allow the actuators 44, 46 to be slid in a proximal or distal direction as desired. For example, the actuators 44, 46 can be moved from the distal position shown in FIG. 1 to the proximal position shown in FIG. 2, and vice versa.

The first actuator 44 includes a manipulation portion 52 that extends in a first direction from the handle 12. The manipulation portion 52, which is preferably configured to be manipulated by hand, but can also be configured to be manipulated by instrument, can be utilized to move the first actuator 44 in a proximal or distal direction within guide channel 48. The first actuator 44 also has a portion 54 that extends through the guide channel 48 of the handle 12 and into cavity 42. The proximal end portion 27 of the first retaining member 20 is attached to the portion 54 of the first actuator 44 so that the first retaining member 20 moves proximally and distally with the first actuator 44

Similar to the first actuator 44, the second actuator 46 can include a manipulation portion 56 that extends in a second direction from the handle 12. The manipulation portion 56, which is preferably configured to be manipulated by hand, but can also be configured to be manipulated by instrument, can be utilized to move the second actuator 46 in a proximal or distal direction within guide channel 50. The second actuator 46 also has a portion 58 that extends through the guide channel 50 in wall 39 of the handle 12 and into cavity 42. The distal end portion 32 of the second retaining member 22 is attached to the portion 58 of the second actuator 46 so that the second retaining member 22 moves proximally and distally with the second actuator 46.

In the illustrated embodiment, the second actuator 46 has a substantially similar configuration to the first actuator 44. However, it should be appreciated that the first and second actuators 44, 46 could each have a configuration different from each other and different from that illustrated in the drawings, depending on the desired use.

To release the medical device 34 from the deployment system 10, referring to FIG. 2, the first actuator 44 is moved, preferably by grasping the manipulation portion 52 by hand, in a proximal direction within channel 48. Movement of the first actuator 44 in the proximal direction causes the first retaining member 20, which is attached to the first actuator, to also move in a proximal direction. Movement of the first retaining member 20 in a proximal direction moves the distal end portion 28 of the first retaining member 20 proximally. In the illustrated embodiment of the deployment system this distal end portion 28 moves out of the opening 30 of the distal end portion 26 of the second retaining member 22.

Once the distal end portion 28 of the first retaining member 20 is moved out of opening 30 in this illustration, the unconstrained second retaining member 22 returns to its flat configuration or is moved away from its upturned configuration so as to enable it to undo its engagement of the medical device. In the illustrated embodiment, the retaining member 22 moves to be substantially out of engagement with the proximal end portion 36 of the medical device 34.

Turning to FIG. 3, to complete the release of the medical device 34 that is illustrated as an embodiment and to reduce the risk of the second retaining member 22 interfering with the release of the medical device 34, the second actuator 46 is moved proximally within channel 50 causing the second retaining member 22 to move proximally and out of the vicinity of the medical device 34, thereby releasing the medical device 34. It will be further appreciated that the proximal movement of the second actuator 46 can directly effect or greatly facilitate movement of the upturned configuration of the distal portion of the second retaining member 22 until same flattens or otherwise moves out of engagement that holds the medical device.

According to one method of delivering the medical device 34, a tubular catheter (not shown) is fed into a body vessel until a distal end thereof is adjacent to a target location. Thereafter, the deployment system 10 and associated attached implantable medical device 34, as illustrated in FIG. 1, are advanced through the catheter, using procedures and techniques generally known in the art, until the device 34 is itself generally adjacent to the target location. Alternatively, the deployment system 10 and associated device 34 may be pre-loaded in the catheter, with the combination being fed through a body vessel to a target location. Other methods of positioning the implantable medical device 34 generally adjacent to a target location may also be practiced without departing from the scope of the present invention.

To more accurately position the engaged device 34, radiopaque markers (not illustrated) may be attached to the carrier member 14 or the device 34 itself.

Referring to FIG. 2, when the engaged device 34 has been properly positioned and oriented, the first actuator 44 and the first retaining member 20, which is attached thereto, are moved in a proximal direction relative to the handle 12 and carrier member 14. As the first retaining member 20 moves in a proximal direction, the distal end portion 28 of the first retaining member 20 comes out of engagement with the opening 30 at the distal end portion 26 of the second retaining 22. The second retaining member 22 returns to its original substantially flat condition and substantially disengages the aperture-containing end portion 36 of the implantable medical device 34.

Turning to FIG. 3, the second actuator 46 and the second retaining member 22, which is attached to the second actuator, are then moved in a proximal direction to ensure that the distal end portion 26 of the second retaining member 22 completely disengages the medical device 34 and does not interfere with the release of the medical device.

When the implantable medical device 30 is disengaged from the deployment system 10, the deployment system 10 may be removed from the patient alone or in conjunction with the catheter.

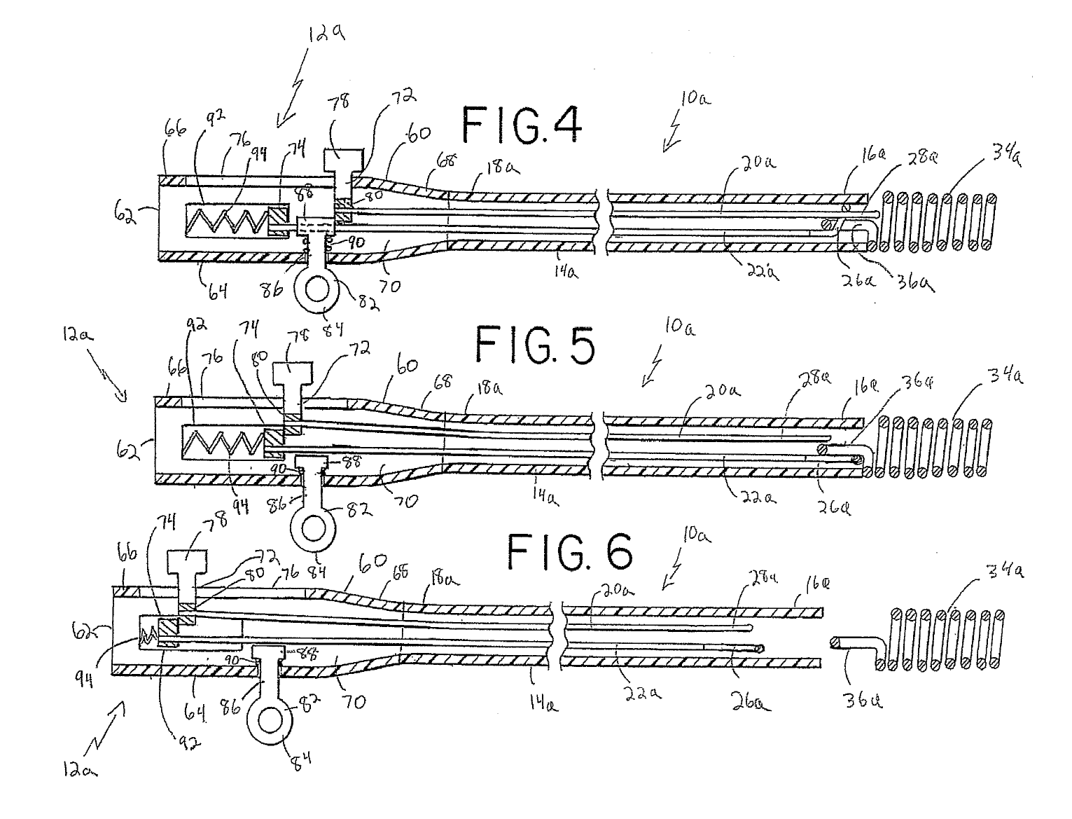

FIGS. 4, 5 and 6 illustrate another embodiment of an actuator handle of the present invention. In this embodiment, the deployment system 10a utilizes actuator handle 12a. The handle 12a is comprised of a handle body 60 including a proximal end wall 62 and a circumferential wall 64. The handle 12a also includes a proximal end portion 66 and a distal end portion 68. The distal end portion 68 of the handle 12a is connected to the proximal end portion 18a of the carrier member 14a. The first and second retaining members 20a, 22a extend past the proximal end portion 18a of the carrier member 14a and into a cavity 70 of the handle 12a.

The handle 12a includes a first actuator 72 and a second actuator 74. The first actuator 72 is slidably connected to a guide channel 76 in wall 64 and can be slid proximally and distally within channel 76 as desired. The first actuator 72 includes a manipulation portion 78 that extends from the handle 12a. The manipulation portion 78, which is preferably configured to be manipulated by hand, but can also be configured to be manipulated by instrument, can be utilized to move the first actuator 72 in a proximal or distal direction within channel 76. The first actuator 72 also has a portion 80 that extends through the channel 70 in wall 64 of the handle 12a and into cavity 70. The first retaining member 20a is attached to the portion 80 of the first actuator 72 so that the first retaining member 20a moves proximally and distally with the first actuator 72.

To prevent premature movement of the first actuator 72, and thus preventing premature release of the medical device, a safety element, such as locking mechanism 82 blocks the first actuator 72 from being slid proximally with channel 76. Illustratively, the locking mechanism 82 can be comprised of a gripping portion 84, a stem 86 and a locking head 88. The stem 86 of the locking mechanism can extend through wall 64 and into cavity 70. The gripping portion 84 is located at one end of the stem 86 and extends outwardly from wall 64. The locking head 88 is located at the other end of the stem 86 and extends into cavity 70. The locking mechanism 82 can be moved between the locked or activated position (FIG. 4) and the unlocked or deactivated position (FIG. 5).

In the activated position, the locking head 88 can be positioned within the cavity 70 to contact the first actuator 72, blocking movement of the actuator 72 in the proximal direction. The locking mechanism 82 can be biased to the locked position by a biasing member 90, such as the illustrated spring positioned around the stem 86 and situated between the locking head 88 and proximal wall 62 of the handle 12a.

To unlock or deactivate the locking mechanism 82, the gripping portion 84 is gripped and pulled in a direction away from the wall 64 of the handle 12a. The locking head 88 moves out of contact with the first actuator 72 and into a position that allows the first actuator to move proximally.

The second actuator 74 is located in a guide track 92 located within the cavity 70 of the handle 12a. The second actuator 74 is movable between a distal position (FIG. 4) and a proximal position (FIG. 6). The second retaining member 22a is connected to the second actuator 74 so that the second retaining member 22a moves proximally and distally along with the second actuator 74. The second actuator 74 can be biased to the distal position by a biasing member 94 located between the second actuator 74 and the proximal wall 62 of the handle 12a.

To initiate release of the medical device 34a connected to the deployment system 10a of FIG. 4, the locking mechanism 82 is deactivated or unlock by grasping the gripping portion 84 and pulling the gripping portion 84 in a direction away from the handle 12a so that the locking head 88 moves out of contact with the first actuator and into a position that allows the first actuator 72 to be moved proximally. Referring to FIG. 5, the first actuator 72 and the first retaining member 20a, which is attached to the first actuator, are moved proximally so that the distal end portion 28a of the first retaining member 20a disengages the distal end portion 28a of the second retaining member 22a, and the first actuator 72 contacts the second actuator 74. The second retaining member 22a returns to its original substantially flat condition and substantially disengages the aperture-containing end portion 36a of the implantable medical device 34a.

Referring to FIG. 6, the first actuator 72 is moved further in the proximal direction, and the contact between the first actuator 72 and the second actuator 74 moves the second actuator and the second retaining member 22a, which is attached to the second actuator, in a proximal direction so that that the distal end portion 26a of the second retaining member 22a completely disengages the proximal end portion 36a of the medical device 34a, and does not interfere with the release of the medical device 34a.

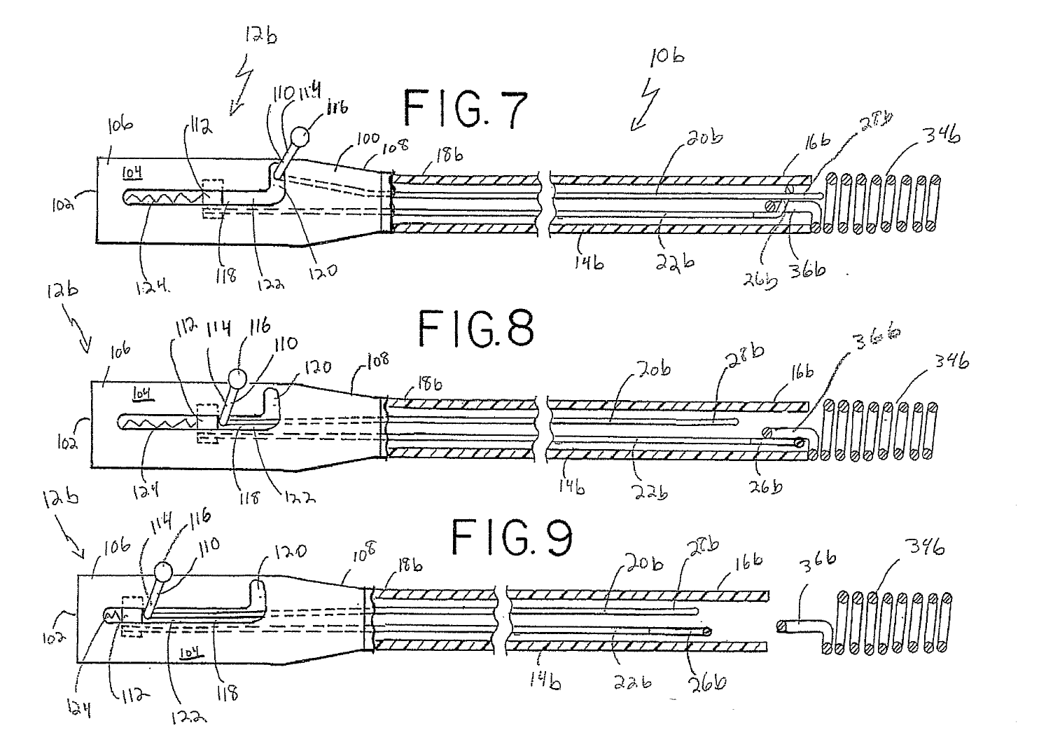

FIGS. 7-9 illustrate another embodiment of an actuator handle of the present invention. In this embodiment, the deployment system 10b utilizes actuator handle 12b, which has a bolt action-like operation. The handle 12b comprises a handle body 100 having a proximal wall 102 and a circumferential wall 104. The handle 12b includes a proximal end portion 106 and a distal end portion 108. The distal end portion 108 of the handle 12b is connected to the proximal end portion 18b of the carrier member 14b. The first and second retaining members 20b, 22b extend past the proximal end portion 18b of the carrier member 14b and into handle 12b.

The handle 12a includes a first actuator 110 and a second actuator 112 (partially shown in phantom). The first actuator 110 includes a post 114 and a gripping portion 116. The post 114 extends through a guide channel 118 that is located in the wall 104 of the handle 12b. The actuator 110 can be slid proximally and distally within the guide channel 118. The shape of the guide channel 118 can provide a safety or locking feature that aids in preventing premature movement of the first actuator 110 in the proximal direction, and thus prevents the premature release of the medical device 34b. For example, in the illustrated embodiment, the guide channel 118 is L-shaped and includes a first section 120 and a second section 122. Prior to actuation of the first actuator 110, the first actuator is located in the first section 120 of the guide channel 118 which allows the first actuator to move in a circumferential direction, but prevents the first actuator 110 from moving in a proximal direction. While in the first section 120, the first actuator 110 can be moved in a circumferential direction into the second section 122 of the channel 118. Once in the second channel 122, the first actuator 110 can be moved in a proximal direction. The first retaining member 20b is attached the post 114 of the first actuator 110 so that the first retaining member 20b moves proximally and distally with the first actuator 110.

The second actuator 112 is located in a guide track (not shown) located within the body 106 of the handle 12b. The second actuator 112 is movable between a distal position (FIG. 7) and a proximal position (FIG. 9). The second retaining member 22b is connected to the second actuator 112 so that the second retaining member 22b moves proximally and distally along with the second actuator 112. The second actuator 112 can be biased to the distal position by a biasing member 124 located between the second actuator 110 and the proximal wall 102 of the handle 12b.

To initiate release of the medical device 34b connected to the deployment system 106 of FIG. 7, the gripping portion 116 is grasped, and the first actuator 110 is moved circumferentially within the first section 120 of the guide channel 118 and into the second section 122 of the guide channel 118. Referring to FIG. 8, the first actuator 110 is moved proximally within section 122 and into contact will second actuator 112. As the first actuator 110 is moved proximally, the first retaining member 20b, which is attached to the first actuator, is also moved proximally. Movement of the retaining member 20b in a proximal direction causes the distal end portion 28b of the first retaining member to disengage the distal end portion 26b of the second retaining member 22b. The second retaining member 22b returns to its original substantially flat condition and substantially disengages the aperture-containing end portion 36b of the implantable medical device 34b.

Referring to FIG. 9, the first actuator 110 is moved further in the proximal direction, and the contact between the first actuator 110 and the second actuator 112 moves the second actuator 112 and the second retaining member 22b, which attached to the second actuator, in a proximal direction to completely release the medical device 34b and reduce the risk of the distal end portion 26b of the second retaining member 22b interfering with the release of the medical device 34b.

It will be understood that the embodiments of the present invention which have been described are illustrative of some of the applications of the principles of the present invention. Numerous modifications may be made by those skilled in the art without departing from the true spirit and scope of the invention, including those combinations of features that are individually disclosed or claimed herein.

1. An handle for use with an implantable medical device deployment system including a first retaining member and a second retaining member wherein manipulation of said first retaining member and said second retaining member controls the release of an implantable medical device from the deployment system, the handle comprising:
a handle body adapted to be connected to the deployment system;
a first actuator slidably connected to the handle body and extending in a first direction from the handle body, said first actuator adapted to be operatively connected to the first retaining member so that the first retaining member can be manipulated by movement of the first actuator; and
a second actuator slidably connected to the handle body and extending in a second direction from the handle body, said second actuator adapted to be operatively connected to the second retaining member so that the second retaining member can be manipulated by movement of the second actuator.

2. The handle of claim 1 in which the first actuator extends in the first direction which is diametrically opposed to the second direction in which the second actuator extends.

3. The handle of claim 1 in which the first actuator is slidably positioned through a guide channel of the handle body.

4. The handle of claim 3 in which the second actuator is slidably positioned through a guide channel of the handle body.

5. The handle of claim 1 in which the first actuator slides in a proximal direction and a distal direction as desired.

6. The handle of claim 5 in which the second actuator slides in a proximal direction and a distal direction as desired.

7. A handle for use with an implantable medical device deployment system including a first retaining member and a second retaining member wherein manipulation of said first retaining member and said second retaining member controls the release of an implantable medical device from the deployment system, the handle comprising:
a handle body adapted to be connected to the deployment system;
a first actuator slidably connected to the handle body, said first actuator adapted to be operatively connected to the first retaining member so that the first retaining member can be manipulated by movement of the first actuator;
a second actuator slidably connected to the handle body, said second actuator adapted to be operatively connected to the second retaining member so that the second retaining member can be manipulated by movement of the second actuator; and
a locking mechanism preventing said first actuator from moving relative to the handle body until the locking mechanism is deactivated.

8. The handle of claim 7 in which the locking mechanism includes a movable portion having an activated position in which said portion prevents movement of the first actuator by contacting the first actuator, and a deactivated position in which said portion is moved out of contact with the first actuator to allow movement of the first actuator.

9. The handle of claim 7 in which the locking mechanism is biased to a locked position by a biasing member.

10. The handle of claim 7 in which the locking mechanism comprises a channel in the handle body.

11. The handle of claim 10 in which the channel is substantially L-shaped.

12. The handle of claim 7 in which the first actuator includes a manipulation portion extending from the body of the handle.

13. The handle of claim 12 in which the second actuator is wholly located within the handle.

14. The handle of claim 12 in which the second actuator is located proximal the first actuator, and the second actuator is moved proximally through contact with the first actuator.

15. The handle of claim 7 in which the second actuator is biased to a distal position by a biasing member.

16. A deployment system for delivering an implantable medical device to a target location of a body vessel, comprising:
a generally elongated hollow carrier member having a proximal end portion and a distal end portion;
a first retaining member extending through the hollow carrier member, said first retaining member having a proximal end portion and a distal end portion;
a second retaining member extending through the hollow carrier member, said second retaining member having a proximal end portion and a distal end portion;
the distal ends of said first and second retaining members cooperating to releasably attach a medical device to the deployment system at or near the distal end portion of the carrier member, manipulation of either of said first retaining member, said second retaining member or both causing the release of the medical device;
a handle having a proximal end portion and a distal end portion, the distal end portion of the handle connected to the proximal end portion of the carrier member;
a first actuator slidably connected to the handle and extending from the handle, said first actuator operatively connected to the first retaining member so that the first retaining member is manipulated by movement of the first actuator; and
a second actuator slidably connected to the handle and extending from the handle, said second actuator operatively connected to the second retaining member so that the second retaining member is manipulated by movement of the second actuator.

17. The deployment system of claim 16 in which the first actuator and the second actuator extend from the handle in different directions.

18. The deployment system of claim 16 in which the first actuator is slidably positioned through a guide channel of the handle.

19. The deployment system of claim 18 in which the second actuator is slidably positioned through a guide channel of the handle.

20. The deployment system of claim 16 in which the first and second actuators slide in a proximal direction or in a distal direction as desired.

21. A deployment system for delivering an implantable medical device to a target location of a body vessel, comprising:
a generally elongated hollow carrier member having a proximal end portion and a distal end portion;
a first retaining member extending through the hollow carrier member, said first retaining member having a proximal end portion and a distal end portion;
a second retaining member extending through the hollow carrier member, said second retaining member having a proximal end portion and a distal end portion;
the distal ends of said first and second retaining members cooperating to releasably attach a medical device to the deployment system at or near the distal end portion of the carrier member, manipulation of either of said first retaining member, said second retaining member or both causing the release of the medical device;
a handle having a proximal end portion and a distal end portion, the distal end portion of the handle connected to the proximal end portion of the carrier member;
a first actuator slidably connected to the handle, said first actuator operatively connected to the first retaining member so that the first retaining member is manipulated by movement of the first actuator; and
a second actuator slidably connected to the handle, said second actuator operatively connected to the second retaining member so that the second retaining member is manipulated by movement of the second actuator; and
a locking mechanism preventing said first actuator from moving until the locking mechanism is deactivated.

22. The deployment system of claim 21 in which the locking mechanism includes a movable portion having an activated position in which the portion prevents movement of the first actuator by contacting the first actuator, and a deactivated position in which the portion is moved out of contact with the first actuator to allow movement of the first actuator.

23. The deployment system of claim 21 in which the locking mechanism is biased to a locked position by a biasing member.

24. The deployment system of claim 21 in which the locking mechanism comprises a channel in the handle.

25. The deployment system of claim 24 in which the channel is substantially L-shaped.

26. The deployment system of claim 21 in which the first actuator includes a manipulation portion extending from the body of the handle.

27. The deployment system of claim 21 in which the second actuator is wholly located within the handle.

28. The deployment system of claim 27 in which the second actuator is located proximal the first actuator, and the second actuator is moved proximally through contact with the first actuator.

29. The deployment system of claim 21 in which the second actuator is biased to a distal position by a biasing member.

30. A method of deploying an implantable medical device to a target location of a body vessel, comprising:
providing a generally elongated hollow carrier member having a proximal end portion and a distal end portion, a first retaining member and a second retaining member each extending through the hollow carrier member, said first and second retaining members cooperating to releasably attach a medical device to the deployment system wherein manipulation of said first and second retaining members effectuate release of the medical device, a handle connected to the proximal end portion of the carrier member, a first actuator slidably connected to the handle, said first actuator connected to the first retaining member for manipulation of the first retaining member, and a second actuator slidably connected to the handle, said second actuator connected to the second retaining member for manipulation of said second retaining member;
positioning the implantable medical device generally adjacent to a target location within the body vessel;
moving the first actuator to manipulate the first retaining member; and
moving the second actuator to manipulate the second retaining member, thereby releasing the medical device.

31. The method of claim 30 in which the moving of the first actuator comprises moving the first actuator in a proximal direction.

32. The method of claim 30 in which the moving of the second actuator comprises moving the second actuator in a proximal direction.

33. The method of claim 30 in which the providing includes having a locking mechanism for preventing the first actuator from moving until the locking mechanism is deactivated; and further including deactivating the locking mechanism prior to said moving the first actuator to manipulate the first retaining member and moving the second actuator to manipulate the second retaining member, thereby releasing the medical device.

34. The method of claim 30 in which moving of the second actuator comprises moving said second actuator through contact with the first actuator.
